(19) United States
(12) Patent Application Publication
(10) Pub. No.: US 2022/0333179 A1
(43) Pub. Date: Oct. 20, 2022

(54) BIOCHIP DETECTION METHOD, DEVICE, AND APPARATUS

(71) Applicant: BOE Technology Group Co., Ltd., Beijing (CN)

(72) Inventors: Mengjun HOU, Beijing (CN); Qiong WU, Beijing (CN); Zongmin LIU, Beijing (CN); Liye DUAN, Beijing (CN)

(21) Appl. No.: 17/631,395

(22) PCT Filed: Mar. 5, 2021
(86) PCT No.: PCT/CN2021/079233

§ 371 Date: Jan. 28, 2022

(30) Foreign Application Priority Data
Mar. 12, 2020 (CN) 202010169754.5

(51) Int. Cl. C12Q 1/6851; B01L 3/00; B01L 7/00; G06T 7/00

(52) U.S. Cl. CPC C12Q 1/6851; B01L 3/502715; B01L 7/52; G06T 7/0012; C12Q 1/686

(57) ABSTRACT

The present disclosure relates to the field of biochip detection, and provides a biochip detection method, a biochip detection device, and an biochip detection apparatus. The biochip detection method includes: introducing a to-be-tested sample into a biochip, the biochip including a plurality of micro-reaction chambers; performing PCR amplification on the to-be-tested sample in the biochip; irradiating the biochip with excitation light rays at different intensities, and collecting images of the biochip under the excitation light rays at different intensities, the excitation light rays being used to excite a fluorescent probe in the to-be-tested sample to emit light; performing data processing on the collected images to obtain the quantity of positive micro-reaction chambers; and calculating the quantity of copies of the to-be-tested sample in accordance with the quantity of positive micro-reaction chambers.

### CROSS-REFERENCE TO RELATED APPLICATIONS

This application claims a priority of the Chinese patent application No. 202010169754.5 filed on Mar. 12, 2020, the entire contents of which are incorporated herein by reference.

### TECHNICAL FIELD

The present disclosure relates to the field of biochip detection, in particular to a biochip detection method, a biochip detection device, and a biochip detection apparatus.

### BACKGROUND

Polymerase Chain Reaction (PCR) has become a new standard in the field of molecular diagnostics. A PCR method relates to the design of primer probes for gene sequences, and the design of primer probes is specific. In the PCR method, the primer probes are detected at a molecular level. As compared with immunological methods, it is able to significantly improve the detection accuracy and sensitivity through the PCR method.

In a digital polymerase chain reaction chip technology (dPCR), a nucleic acid sample is diluted sufficiently, so as to enable the quantity of sample templates in each micro-reaction chamber to be less than or equal to 1, thereby to realize absolute quantification analysis on a single molecule DNA. The dPCR has been widely used in clinical diagnosis, gene instability analysis, single cell gene expression, detection of environmental microorganisms, and prenatal diagnosis due to such advantages as high sensitivity, specificity, high detection flux and quantitative accuracy.

Fluorescence image acquisition and sample point identification are required after the amplification of a dPCR chip array. Existing biochip sample point detection methods mainly include a parameter-dependent method, a marker-assisted method, and an automatic detection method. However, the quantity of micro-reaction chambers needs to be obtained through setting a signal intensity threshold, so high chip imaging quality and uniformity are demanded. If the chip imaging quality and uniformity fail to meet the requirement, the detection accuracy will be adversely affected.

### SUMMARY

The present disclosure provides the following technical solutions.

In one aspect, the present disclosure provides in some embodiments a biochip detection method, including: introducing a to-be-tested sample into a biochip, the biochip including a plurality of micro-reaction chambers; performing PCR amplification on the to-be-tested sample in the biochip; irradiating the biochip with excitation light rays at different intensities, and collecting images of the biochip under the excitation light rays at different intensities, the excitation light rays being used to excite a fluorescent probe in the to-be-tested sample to emit light; performing data processing on the collected images to obtain the quantity of positive micro-reaction chambers; and calculating the quantity of copies of the to-be-tested sample in accordance with the quantity of positive micro-reaction chambers.

In a possible embodiment of the present disclosure, the irradiating the biochip with the excitation light rays at different intensities and collecting the images of the biochip under the excitation light rays at different intensities includes controlling the intensity of the excitation light rays to increase linearly from 0 to A within a preset time period T, and collecting an image every T/(N−1) to obtain N images totally. An exposure time of each image is the same, and N is an integer greater than 1.

In a possible embodiment of the present disclosure, A is a maximum intensity of the excitation light ray which is capable of being accepted by the fluorescent probe in the to-be-tested sample.

In a possible embodiment of the present disclosure, the performing data processing on the collected images to obtain the quantity of positive micro-reaction chambers includes: obtaining information about a central position of each micro-reaction chamber in the N images; determining pixels for each micro-reaction chamber in the N images in accordance with a size of each micro-reaction chamber and the information about the central position of each micro-reaction chamber; determining a fluorescence intensity of each micro-reaction chamber in the N images in accordance with a grayscale value of each pixel for each micro-reaction chamber; and determining the quantity of positive micro-reaction chambers in accordance with the fluorescence intensity of each micro-reaction chamber in the N images.

In a possible embodiment of the present disclosure, the obtaining the information about the central position of each micro-reaction chamber in the N images includes: binarizing an N^{th} image to obtain a binary image; performing a morphological dilation operation on the binary image in accordance with a first dilation operator in a row direction to obtain a first image, a connected domain in the row direction in the first image representing a row of micro-reaction chambers; performing a morphological dilation operation on the binary image in accordance with a second dilation operator in a column direction to obtain a second image, a connected domain in the column direction in the second image representing a column of micro-reaction chambers; detecting connected domains in the row direction in the first image so as to determine the quantity of rows of an array of micro-reaction chambers and information about a central position of each micro-reaction chamber in each row in the column direction; detecting connected domains in the column direction in the second image so as to determine the quantity of columns of an array of micro-reaction chambers and information about a central position of each micro-reaction chamber in each column in the row direction; and obtaining the information about the central position of each micro-reaction chamber in accordance with the information about the central position of each micro-reaction chamber in each row in the column direction and the information about the central position of each micro-reaction chamber in each column in the row direction.

In a possible embodiment of the present disclosure, the fluorescence intensity of the micro-reaction chamber is an average of the grayscale values of all pixels for the micro-reaction chambers.

In a possible embodiment of the present disclosure, the determining the quantity of positive micro-reaction chambers in accordance with the fluorescence intensity of each micro-reaction chamber in the N images includes determining a fluorescence intensity curve of each micro-reaction chamber in accordance with the fluorescence intensity of each micro-reaction chamber in the N images, and determining a micro-reaction chamber whose fluorescence intensity is positively correlated to the intensity of excitation light ray as the positive reaction micro-reaction chamber.

In a possible embodiment of the present disclosure, the binarizing the N^{th} image to obtain the binary image includes binarizing the N^{th} image using an Otsu algorithm to obtain the binary image.

In a possible embodiment of the present disclosure, the calculating the quantity of copies of the to-be-tested sample in accordance with the quantity of positive micro-reaction chambers includes calculating the quantity of copies of the to-be-tested sample through c=[ln(1−f/n)]/m, where n is the total quantity of micro-reaction chambers, f is the quantity of positive micro-reaction chambers, m is a dilution factor of the to-be-tested sample, and c is the quantity of copies of the to-be-tested sample.

In another aspect, the present disclosure provides in some embodiments a biochip detection device, including: an introduction module configured to introduce a to-be-tested sample into a biochip, the biochip including a plurality of micro-reaction chambers; an amplification module configured to perform PCR amplification on the to-be-tested sample in the biochip; an image collection module configured to irradiate the biochip with excitation light rays at different intensities, and collect images of the biochip under the excitation light rays at different intensities, the excitation light rays being used to excite a fluorescent probe in the to-be-tested sample to emit light; a data processing module configured to perform data processing on the collected images to obtain the quantity of positive micro-reaction chambers; and a calculation module configured to calculate the quantity of copies of the to-be-tested sample in accordance with the quantity of positive micro-reaction chambers.

In yet another aspect, the present disclosure provides in some embodiments a biochip detection apparatus, including a memory and a processor. A computer program is stored in the memory and executed by a processor so as to implement steps of the above-mentioned biochip detection method.

In still yet another aspect, the present disclosure provides in some embodiments a computer-readable medium storing therein a computer program. The computer program is executed by a processor so as to implement steps of the above-mentioned biochip detection method.

### BRIEF DESCRIPTION OF THE DRAWINGS

FIG. 1 is a flow chart of a biochip detection method according to one embodiment of the present disclosure;

FIG. 2 is a top view of a biochip according to one embodiment of the present disclosure;

FIG. 3 is a schematic view showing a biochip detection device according to one embodiment of the present disclosure; and

FIG. 4 is a schematic view showing components of a biochip detection apparatus according to one embodiment of the present disclosure.

### DETAILED DESCRIPTION

In order to make the objects, the technical solutions and the advantages of the present disclosure more apparent, the present disclosure will be described hereinafter in a clear and complete manner in conjunction with the drawings and embodiments.

In the related art, when determining the quantity of positive micro-reaction chambers, a fluorescence image of a biochip is collected, a signal intensity threshold value is set, and a micro-reaction chamber with a fluorescence intensity greater than the signal intensity threshold value is determined as a positive micro-reaction chamber. However, in this method, the imaging quality and uniformity of the biochip are highly demanded, and if the imaging quality and uniformity of the biochip fail to meet the requirement, the detection accuracy will be adversely affected.

An object of the present disclosure is to provide a biochip detection method, a biochip detection device, and a biochip detection apparatus so as to improve the detection accuracy of a biochip.

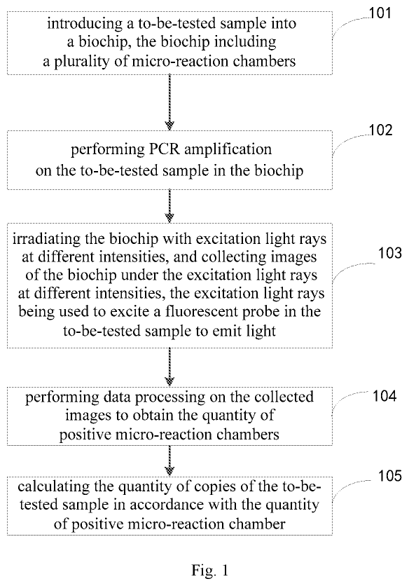

The present disclosure provides in some embodiments a biochip detection method which, as shown in FIG. 1, includes the following steps.

Step 101: introducing a to-be-tested sample into a biochip which includes a plurality of micro-reaction chambers.

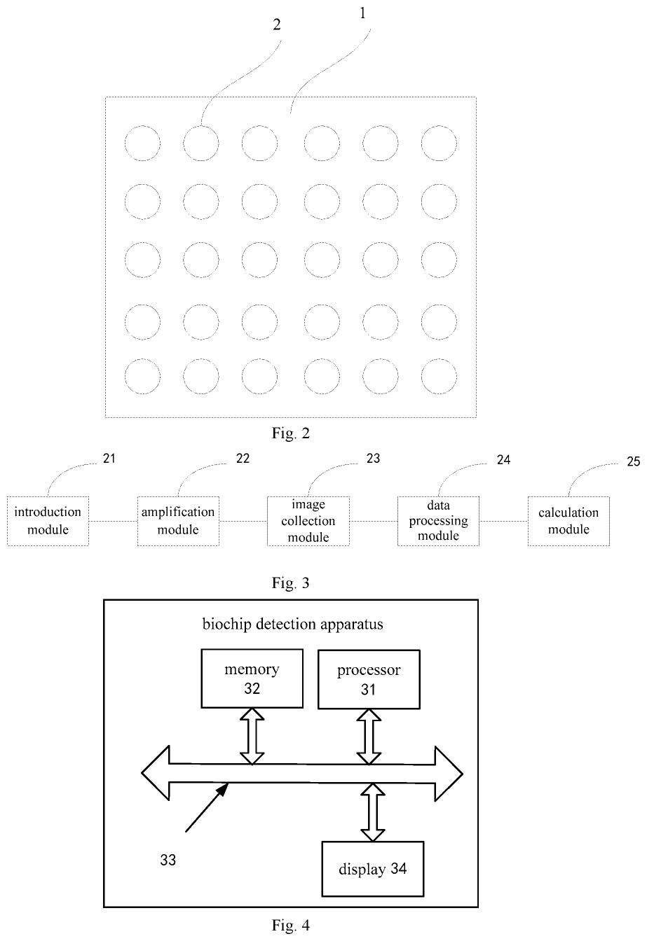

As shown in FIG. 2, the biochip 1 includes a plurality of micro-reaction chambers 2 arranged in an array form. The to-be-tested sample is introduced into the biochip 1 in such a manner as to be distributed in the plurality of micro-reaction chambers 2. To be specific, the to-be-tested sample may be a sample solution including a nucleic acid.

Step 102: performing PCR amplification on the to-be-tested sample in the biochip.

To be specific, the biochip may be heated to subject the to-be-tested sample to thermal cycling amplification.

Step 103: irradiating the biochip with excitation light rays at different intensities, and collecting images of the biochip under the excitation light rays at different intensities, the excitation light rays being used to excite a fluorescent probe in the to-be-tested sample to emit light.

To be specific, the intensity of the excitation light rays may be controlled to increase linearly from 0 to A within a preset time period T, and an image may be obtained every T/(N−1) to obtain totally N images. Specifically, a first image, a second image, . . . , and an N^{th} image are obtained at time points 0, T/(N−1), 2T/(N−1), . . . , and T respectively, an exposure time of each image is the same, and N is an integer greater than 1.

During the collection of the first image, the intensity of the excitation light ray is 0, and during the collection of the N^{th} image, the intensity of the excitation light ray is A, where A is a maximum intensity of the excitation light ray which is capable of being accepted by the fluorescent probe in the to-be-tested sample. Of course, A may also be less than the maximum intensity of the excitation light ray. In this way, the N images correspond to the excitation light rays at different intensities, and under the excitation of the excitation light rays at different intensities, the fluorescent probe in the to-be-tested sample may emit fluorescent light at different intensities.

In the embodiments of the present disclosure, the intensity of the excitation light ray may also be controlled to increase linearly from B to A within the preset time period T, and one image may be collected every T/(N−1) so as to obtain totally N images. Specifically, a first image, a second image . . . , and an N^{th} image are obtain at time points 0, T/(N−1), 2T/(N−1), . . . , and T respectively, an exposure time of each image is the same, and B is less than A.

Step 104: performing data processing on the collected images to obtain the quantity of positive micro-reaction chambers.

Step 105: calculating the quantity of copies of the to-be-tested sample in accordance with the quantity of positive micro-reaction chambers.

According to the embodiments of the present disclosure, after performing PCR amplification on the to-be-tested sample in the biochip, the biochip is irradiated with the excitation light rays at different intensities, the images of the biochip are collected under the excitation light rays at different intensities, the data processing is performed on the collected images to obtain the quantity of positive micro-reaction chambers, and then the quantity of copies of the to-be-tested sample is calculated. Different from a scheme where the quantity of positive micro-reaction chambers is determined in accordance with an individual image through setting a threshold, in the embodiments of the present disclosure, a plurality of images is collected under the excitation light rays at different intensities, and then processed to obtain the quantity of positive micro-reaction chambers. As a result, it is able to improve the detection accuracy of the biochip.

In some embodiments of the present disclosure, the performing data processing on the collected images to obtain the quantity of positive micro-reaction chambers includes: obtaining information about a central position of each micro-reaction chamber in the N images; determining pixels for each micro-reaction chamber in the N images in accordance with a size of each micro-reaction chamber and the information about the central position of each micro-reaction chamber (after collecting the image of the biochip, the size of the micro-reaction chamber in the image may be determined through determining a boundary of the micro-reaction chamber, and then the pixels for each micro-reaction chamber in the image may be determined in accordance with the information about the central position of the micro-reaction chamber); determining a fluorescence intensity of each micro-reaction chamber in the N images in accordance with a grayscale value of each pixel for each micro-reaction chamber; and determining the quantity of positive micro-reaction chambers in accordance with the fluorescence intensity of each micro-reaction chamber in the N images.

In some embodiments of the present disclosure, the obtaining the information about the central position of each micro-reaction chamber in the N images includes: binarizing an N^{th} image to obtain a binary image; performing a morphological dilation operation on the binary image in accordance with a first dilation operator in a row direction to obtain a first image, a connected domain in the row direction in the first image representing a row of micro-reaction chambers; performing a morphological dilation operation on the binary image in accordance with a second dilation operator in a column direction to obtain a second image, a connected domain in the column direction in the second image representing a column of micro-reaction chambers; detecting connected domains in the row direction in the first image so as to determine the quantity of rows of an array of micro-reaction chambers and information about a central position of each micro-reaction chamber in each row in the column direction; detecting connected domains in the column direction in the second image so as to determine the quantity of columns of an array of micro-reaction chambers and information about a central position of each micro-reaction chamber in each column in the row direction; and obtaining the information about the central position of each micro-reaction chamber in accordance with the information about the central position of each micro-reaction chamber in each row in the column direction and the information about the central position of each micro-reaction chamber in each column in the row direction.

There merely exist two states for a brightness value of the binary image, i.e., black (0) and white (255).

The N^{th} image is an image of the biochip collected under the excitation light ray at a maximum intensity. The larger the intensity of the excitation light ray, the larger the fluorescence intensity of the fluorescent probe in the to-be-tested sample. Hence, the N^{th} image is an image with the largest fluorescence intensity. The information about the central position of each micro-reaction chamber may be obtained in accordance with the N^{th} image, so it is able to improve the accuracy of the information about the central position.

To be specific, the N^{th} image may be binarized using an Otsu algorithm to obtain the binary image. A threshold T1 for the binarization may be obtained through the Otsu algorithm. However, a binarization algorithm will not be particularly defined herein, and in some other embodiments of the present disclosure, any other binarization algorithm may be adopted, or a default threshold may be used for the binarization.

In the embodiments of the present disclosure, each micro-reaction chamber may be of a circular or rectangular shape, which will not be particularly defined herein.

Morphological dilation is used to enlarge an object of interest in an image. For example, when an original picture is a smiling face, the dilation is used to thicken an outline of the smiling face in the image.

Generally, the connected domain refers to an image region consisting of foreground pixel points having a same grayscale value and adjacent to each other in the image. Illustratively, a value of the first dilation operator in the row direction may be a width of the image of the biochip, and a value of the second dilation operator in the column direction may be a height of the image of the biochip. Furthermore, a value of the first dilation operator in the column direction may be 1, and a value of the second dilation operator in the row direction may be 1. However, the values of the first dilation operator and the second dilation operator will not be particularly defined herein.

The information about the central position may include coordinates of the central position in a coordinate system. The information about the central position of each micro-reaction chamber in each row in the column direction may be stored in a one-dimensional vector, and a length of the one-dimensional vector is just the quantity of rows of an array of micro-reaction chambers. The information about the central position of each micro-reaction chamber in each column in the row direction may be stored in a one-dimensional vector, and a length of the one-dimensional vector is just the quantity of columns of the array of micro-reaction chambers. However, the present disclosure is not limited thereto. In some other embodiments of the present disclosure, the quantity of rows of the array of micro-reaction chambers and the information about the central position of each micro-reaction chamber in each row in the column direction may be stored in a two-dimensional vector. For example, row indices and the corresponding information about the central position may be recorded in the two-dimensional vector.

Illustratively, the detecting the connected domains in the first image in the row direction may be detected so as to determine the quantity of rows in the array of micro-reaction chambers and the information about the central position of each micro-reaction chamber in each row in the column direction may include obtaining the quantity of rows in the array of micro-reaction chambers and the information about the central position of each micro-reaction chamber in each row in the column direction in accordance with the first image using a findContours function in an Open Source Computer Vision Library (OpenCV).

The detecting the connected domains in the second image in the column direction so as to determine the quantity of columns in the array of micro-reaction chambers and the information about the central position of each micro-reaction chamber in each column in the row direction may include obtaining the quantity of columns in the array of micro-reaction chambers and the information about the central position of each micro-reaction chamber in each column in the row direction in accordance with the second image using the findContours function in OpenCV.

In the embodiments of the present disclosure, through the findContours functions in OpenCV, it is able to simply the algorithm implementation. However, the present embodiment is not limited thereto. In some other embodiments of the present disclosure, any other known edge detection algorithm or a customized edge detection algorithm may be adopted to detect the connected domain.

In a possible embodiment of the present disclosure, an average of the grayscale values of all the pixels for the micro-reaction chamber may be taken as the fluorescence intensity of the micro-reaction chamber. For example, when coordinates of a central position of a micro-reaction chamber are (x, y), P (x, y) is a grayscale value of the central position of the micro-reaction chamber and the micro-reaction chamber includes 9 pixels arranged in an array form, i.e., (x−1, y−1), (x, y−1), (x+1, y−1), (x−1, y), (x, y), (x+1, y), (x−1, y+1), (x, y+1), (x+1, y+1), the fluorescence intensity of the micro-reaction chamber may be [P (x−1, y−1)+P (x, y−1)+P (x+1, y−1)+P (x−1, y)+P (x, y)+P (x+1, y)+P (x−1, y+1)+P (x, y+1)+P (x+1, y+1), Y+1)/9, where P (x−1, y−1), P (x+1, y+1) are grayscale values at corresponding positions.

Of course, apart from taking the average of the grayscale values of all the pixels for the micro-reaction chamber as the fluorescence intensity of the micro-reaction chamber, in the embodiments of the present disclosure, the fluorescence intensity of the micro-reaction chamber may be determined using any other methods. For example, several pixels for the micro-reaction chamber with largest grayscale values may be selected, and an average of the grayscale values of these pixels may be taken as the fluorescence intensity of the micro-reaction chamber. In this way, it is able to reduce a computational burden.

In some embodiments of the present disclosure, the determining the quantity of positive micro-reaction chambers in accordance with the fluorescence intensity of each micro-reaction chamber in the N images includes determining a fluorescence intensity curve of each micro-reaction chamber in accordance with the fluorescence intensity of each micro-reaction chamber in the N images, and determining a micro-reaction chamber whose fluorescence intensity is positively correlated to the intensity of excitation light ray as the positive reaction micro-reaction chamber.

When the N images are processed as mentioned hereinabove, N fluorescence intensities of each micro-reaction chamber in the N images may be obtained. The fluorescence intensity of the micro-reaction chamber and the intensity of the corresponding excitation light ray may form a group of data, so N groups of data about each micro-reaction chamber may be obtained. Then, a curve may be fitted in accordance with the N groups of data, so as to obtain the fluorescence intensity curve of each micro-reaction chamber. In the fluorescence intensity curve of the positive micro-reaction chamber, the fluorescence intensity is obviously positively correlated to the intensity of the excitation light ray. However, for a negative micro-reaction chamber, the fluorescence intensity is not obviously correlated to the intensity of the excitation light ray. Based on this feature, it is able to determine whether each micro-reaction chamber is a positive micro-reaction chamber in accordance with the fluorescence intensity curve of the micro-reaction chamber, and then determine the quantity f of positive micro-reaction chambers.

In some embodiments of the present disclosure, the calculating the quantity of copies of the to-be-tested sample in accordance with the quantity of positive micro-reaction chambers includes calculating the quantity of copies of the to-be-tested sample through c=[ln(1−f/n)]/m, where n is the total quantity of micro-reaction chambers, f is the quantity of positive micro-reaction chambers, m is a dilution factor of the to-be-tested sample, and c is the quantity of copies of the to-be-tested sample.

The present disclosure further provides in some embodiments a biochip detection device which, as shown in FIG. 3, includes: an introduction module 21 configured to introduce a to-be-tested sample into a biochip, the biochip including a plurality of micro-reaction chambers; an amplification module 22 configured to perform PCR amplification on the to-be-tested sample in the biochip; an image collection module 23 configured to irradiate the biochip with excitation light rays at different intensities, and collect images of the biochip under the excitation light rays at different intensities, the excitation light rays being used to excite a fluorescent probe in the to-be-tested sample to emit light; a data processing module 24 configured to perform data processing on the collected images to obtain the quantity of positive micro-reaction chambers; and a calculation module 25 configured to calculate the quantity of copies of the to-be-tested sample in accordance with the quantity of positive micro-reaction chambers.

As shown in FIG. 2, the biochip 1 includes a plurality of micro-reaction chambers 2 arranged in an array form. The to-be-tested sample is introduced into the biochip 1 in such a manner as to be distributed in the plurality of micro-reaction chambers 2. To be specific, the to-be-tested sample may be a sample solution including a nucleic acid.

To be specific, the biochip may be heated to subject the to-be-tested sample to thermal cycling amplification.

To be specific, the intensity of the excitation light rays may be controlled to increase linearly from 0 to A within a preset time period T, and an image may be obtained every T/(N−1) to obtain totally N images. Specifically, a first image, a second image, . . . , and an N^{th} image are obtained at time points 0, T/(N−1), 2T/(N−1), . . . , and T respectively, an exposure time of each image is the same, and N is an integer greater than 1.

During the collection of the first image, the intensity of the excitation light ray is 0, and during the collection of the N^{th} image, the intensity of the excitation light ray is A, where A is a maximum intensity of the excitation light ray which is capable of being accepted by the fluorescent probe in the to-be-tested sample. Of course, A may also be less than the maximum intensity of the excitation light ray. In this way, the N images correspond to the excitation light rays at different intensities, and under the excitation of the excitation light rays at different intensities, the fluorescent probe in the to-be-tested sample may emit fluorescent light at different intensities.

In the embodiments of the present disclosure, the intensity of the excitation light ray may also be controlled to increase linearly from B to A within the preset time period T, and one image may be collected every T/(N−1) so as to obtain totally N images. Specifically, a first image, a second image . . . , and an N^{th} image are obtain at time points 0, T/(N−1), 2T/(N−1), . . . , and T respectively, an exposure time of each image is the same, and B is less than A.

According to the embodiments of the present disclosure, after performing PCR amplification on the to-be-tested sample in the biochip, the biochip is irradiated with the excitation light rays at different intensities, the images of the biochip are collected under the excitation light rays at different intensities, the data processing is performed on the collected images to obtain the quantity of positive micro-reaction chambers, and then the quantity of copies of the to-be-tested sample is calculated. Different from a scheme where the quantity of positive micro-reaction chambers is determined in accordance with an individual image through setting a threshold, in the embodiments of the present disclosure, a plurality of images is collected under the excitation light rays at different intensities, and then processed to obtain the quantity of positive micro-reaction chambers. As a result, it is able to improve the detection accuracy of the biochip. The biochip detection device in the embodiments of the present disclosure is used to implement the above-mentioned biochip detection method with a same technical effect.

In some embodiments of the present disclosure, the data processing module 24 is specifically configured to: obtain information about a central position of each micro-reaction chamber in the N images; determine pixels for each micro-reaction chamber in the N images in accordance with a size of each micro-reaction chamber and the information about the central position of each micro-reaction chamber; determine a fluorescence intensity of each micro-reaction chamber in the N images in accordance with a grayscale value of each pixel for each micro-reaction chamber; and determine the quantity of positive micro-reaction chambers in accordance with the fluorescence intensity of each micro-reaction chamber in the N images.

In some embodiments of the present disclosure, the data processing module 24 is specifically configured to: binarize an N^{th} image to obtain a binary image; perform a morphological dilation operation on the binary image in accordance with a first dilation operator in a row direction to obtain a first image, a connected domain in the row direction in the first image representing a row of micro-reaction chambers; perform a morphological dilation operation on the binary image in accordance with a second dilation operator in a column direction to obtain a second image, a connected domain in the column direction in the second image representing a column of micro-reaction chambers; detect connected domains in the row direction in the first image so as to determine the quantity of rows of an array of micro-reaction chambers and information about a central position of each micro-reaction chamber in each row in the column direction; detect connected domains in the column direction in the second image so as to determine the quantity of columns of an array of micro-reaction chambers and information about a central position of each micro-reaction chamber in each column in the row direction; and obtain the information about the central position of each micro-reaction chamber in accordance with the information about the central position of each micro-reaction chamber in each row in the column direction and the information about the central position of each micro-reaction chamber in each column in the row direction.

The N^{th} image is an image of the biochip collected under the excitation light ray at a maximum intensity. The larger the intensity of the excitation light ray, the larger the fluorescence intensity of the fluorescent probe in the to-be-tested sample. Hence, the N^{th} image is an image with the largest fluorescence intensity. The information about the central position of each micro-reaction chamber may be obtained in accordance with the N^{th} image, so it is able to improve the accuracy of the information about the central position. To be specific, the N^{th} image may be binarized using an Otsu algorithm to obtain the binary image. A threshold T1 for the binarization may be obtained through the Otsu algorithm. However, a binarization algorithm will not be particularly defined herein, and in some other embodiments of the present disclosure, any other binarization algorithm may be adopted, or a default threshold may be used for the binarization.

In the embodiments of the present disclosure, each micro-reaction chamber may be of a circular or rectangular shape, which will not be particularly defined herein.

Illustratively, a value of the first dilation operator in the row direction may be a width of the image of the biochip, and a value of the second dilation operator in the column direction may be a height of the image of the biochip. Furthermore, a value of the first dilation operator in the column direction may be 1, and a value of the second dilation operator in the row direction may be 1. However, the values of the first dilation operator and the second dilation operator will not be particularly defined herein.

The information about the central position may include coordinates of the central position in a coordinate system. The information about the central position of each micro-reaction chamber in each row in the column direction may be stored in a one-dimensional vector, and a length of the one-dimensional vector is just the quantity of rows of an array of micro-reaction chambers. The information about the central position of each micro-reaction chamber in each column in the row direction may be stored in a one-dimensional vector, and a length of the one-dimensional vector is just the quantity of columns of the array of micro-reaction chambers. However, the present disclosure is not limited thereto. In some other embodiments of the present disclosure, the quantity of rows of the array of micro-reaction chambers and the information about the central position of each micro-reaction chamber in each row in the column direction may be stored in a two-dimensional vector. For example, row indices and the corresponding information about the central position may be recorded in the two-dimensional vector.

Illustratively, the detecting the connected domains in the first image in the row direction may be detected so as to determine the quantity of rows in the array of micro-reaction chambers and the information about the central position of each micro-reaction chamber in each row in the column direction may include obtaining the quantity of rows in the array of micro-reaction chambers and the information about the central position of each micro-reaction chamber in each row in the column direction in accordance with the first image using a findContours function in an OpenCV.

The detecting the connected domains in the second image in the column direction so as to determine the quantity of columns in the array of micro-reaction chambers and the information about the central position of each micro-reaction chamber in each column in the row direction may include obtaining the quantity of columns in the array of micro-reaction chambers and the information about the central position of each micro-reaction chamber in each column in the row direction in accordance with the second image using the findContours function in OpenCV.

In the embodiments of the present disclosure, through the findContours functions in OpenCV, it is able to simply the algorithm implementation. However, the present embodiment is not limited thereto. In some other embodiments of the present disclosure, any other known edge detection algorithm or a customized edge detection algorithm may be adopted to detect the connected domain.

In a possible embodiment of the present disclosure, an average of the grayscale values of all the pixels for the micro-reaction chamber may be taken as the fluorescence intensity of the micro-reaction chamber. For example, when coordinates of a central position of a micro-reaction chamber are (x, y), P (x, y) is a grayscale value of the central position of the micro-reaction chamber and the micro-reaction chamber includes 9 pixels arranged in an array form, i.e., (x−1, y−1), (x, y−1), (x+1, y−1), (x−1, y), (x, y), (x+1, y), (x−1, y+1), (x, y+1), (x+1, y+1), the fluorescence intensity of the micro-reaction chamber may be [P (x−1, y−1)+P (x, y−1)+P (x+1, y−1)+P (x−1, y)+P (x, y)+P (x+1, y)+P (x−1, y+1)+P (x, y+1)+P (x+1, y+1), Y+1)/9, where P (x−1, y−1), P (x+1, y+1) are grayscale values at corresponding positions.

Of course, apart from taking the average of the grayscale values of all the pixels for the micro-reaction chamber as the fluorescence intensity of the micro-reaction chamber, in the embodiments of the present disclosure, the fluorescence intensity of the micro-reaction chamber may be determined using any other methods. For example, several pixels for the micro-reaction chamber with largest grayscale values may be selected, and an average of the grayscale values of these pixels may be taken as the fluorescence intensity of the micro-reaction chamber. In this way, it is able to reduce a computational burden.

In some embodiments of the present disclosure, the data processing module 24 is specifically configured to determine a fluorescence intensity curve of each micro-reaction chamber in accordance with the fluorescence intensity of each micro-reaction chamber in the N images, and determining a micro-reaction chamber whose fluorescence intensity is positively correlated to the intensity of excitation light ray as the positive reaction micro-reaction chamber.

When the N images are processed as mentioned hereinabove, N fluorescence intensities of each micro-reaction chamber in the N images may be obtained. The fluorescence intensity of the micro-reaction chamber and the intensity of the corresponding excitation light ray may form a group of data, so N groups of data about each micro-reaction chamber may be obtained. Then, a curve may be fitted in accordance with the N groups of data, so as to obtain the fluorescence intensity curve of each micro-reaction chamber. In the fluorescence intensity curve of the positive micro-reaction chamber, the fluorescence intensity is obviously positively correlated to the intensity of the excitation light ray. However, for a negative micro-reaction chamber, the fluorescence intensity is not obviously correlated to the intensity of the excitation light ray. Based on this feature, it is able to determine whether each micro-reaction chamber is a positive micro-reaction chamber in accordance with the fluorescence intensity curve of the micro-reaction chamber, and then determine the quantity f of positive micro-reaction chambers.

In some embodiments of the present disclosure, the calculation module 25 is specifically configured to calculate the quantity of copies of the to-be-tested sample through c=[ln(1−f/n)]/m, where n is the total quantity of micro-reaction chambers, f is the quantity of positive micro-reaction chambers, m is a dilution factor of the to-be-tested sample, and c is the quantity of copies of the to-be-tested sample.

The present disclosure further provides in some embodiments a biochip detection apparatus, which includes a memory and a processor. A computer program is stored in the memory and executed by a processor so as to implement steps of the above-mentioned biochip detection method.

As shown in FIG. 4, in one instance, the biochip detection apparatus includes a processor 31, a memory 32, a bus system 33 and a display 34. The processor 31, the memory 32 and the display 34 are coupled to each other via the bus system 33, the memory 32 is configured to store therein instructions, and the processor 31 is configured to execute the instructions in the memory 32 to control a display content of the display 34.

It should be appreciated that, the processor 31 may be a Central Processing Unit (CPU). It may also be a general-purpose processor, a digital signal processor, an application-specific integrated circuit (ASIC), a field programmable gate array (FPGA) or any other programmable logic element, a discrete gate or transistor logic element, or a discrete hardware assembly. The general purpose processor may be a microprocessor or any other conventional processor.

The memory 32 may include read-only memory and random access memory, and it may provide instructions and data to the processor 31. A part of memory 32 may also include non-volatile random access memory. For example, the memory 32 may also store therein device type information.

The bus system 33 may include, in addition to a data bus, a power bus, a control bus, a status signal bus, etc. However, for clarification, various buses in FIG. 4 are marked as the bus system 33.

During the implementation, functions of the data processing device may be implemented by an integrated logic circuit of hardware in the processor 31 or instructions in the form of software, the processor in the form of hardware. In other words, the steps of the method in the embodiments of the present disclosure may be directly implemented by the processor in the form of hardware, or a combination of hardware and software modules in the processor. The software module may be located in a storage medium such as a Random Access Memory (RAM), a flash memory, a Read Only Memory (ROM), a Programmable ROM (PROM), an Electrically Erasable PROM (EEPROM), or a register. The storage medium may be located in the memory 32, and the processor 31 may read information stored in the memory 32 so as to implement the steps of the above-mentioned method, which will not be particularly defined herein.

The present disclosure further provides in some embodiments a computer-readable medium storing therein a computer program. The computer program is executed by a processor so as to implement steps of the above-mentioned biochip detection method.

It should be appreciated that, all or some steps in the method, and functional modules/units in the system and device may be implemented as software, firmware, hardware or a combination thereof. For the hardware implementation, the functional modules/units may not necessarily be divided in such a manner as to correspond to physical components. For example, one physical component may have a plurality of functions, or one function or step may be executed by several physical components. Some or all components may be implemented as software to be executed by a processor, e.g., a DSP or a microprocessor, or implemented as hardware, or implemented as an integrated circuit, e.g., a specific integrated circuit. The software may be located on a computer-readable medium. The computer-readable medium may include a computer-readable storage medium (or non-transient medium) and a communication medium (or transient medium). As is known to a person skilled in the art, the computer-readable storage medium includes any volatile or non-volatile, mobile or immobile medium implemented in a method or technology for storing information (e.g., computer-readable instructions, data structures, program modules or any other data). The computer-readable storage medium includes, but not limited to, an RAM, an ROM, an EEPROM, a flash memory or the like, a CD-ROM, a DVD or the like, a magnetic cassette, a magnetic tape and a magnetic disk, or any other medium for storing therein desired information and accessible to a computer. In addition, usually the communication medium includes a computer-readable instruction, a data structure, a program module or the other data in a carrier or the other modulated data signal such as a transmission mechanism, and it may include any information delivery medium.

The above embodiments are for illustrative purposes only, but the present disclosure is not limited thereto. Obviously, a person skilled in the art may make further modifications and improvements without departing from the spirit of the present disclosure, and these modifications and improvements shall also fall within the scope of the present disclosure.

1. A biochip detection method, comprising:
introducing a to-be-tested sample into a biochip, the biochip comprising a plurality of micro-reaction chambers;
performing Polymerase Chain Reaction (PCR) amplification on the to-be-tested sample in the biochip;
irradiating the biochip with excitation light rays at different intensities, and collecting images of the biochip under the excitation light rays at different intensities, the excitation light rays being used to excite a fluorescent probe in the to-be-tested sample to emit light;
performing data processing on the collected images to obtain the quantity of positive micro-reaction chambers; and
calculating the quantity of copies of the to-be-tested sample in accordance with the quantity of positive micro-reaction chambers.

2. The biochip detection method according to claim 1, wherein the irradiating the biochip with the excitation light rays at different intensities and collecting the images of the biochip under the excitation light rays at different intensities comprises controlling the intensity of the excitation light rays to increase linearly from 0 to A within a preset time period T, and collecting an image every T/(N−1) to obtain N images totally, wherein an exposure time of each image is the same, and N is an integer greater than 1.

3. The biochip detection method according to claim 2, wherein A is a maximum intensity of the excitation light ray which is capable of being accepted by the fluorescent probe in the to-be-tested sample.

4. The biochip detection method according to claim 2, wherein the performing data processing on the collected images to obtain the quantity of positive micro-reaction chambers comprises:
obtaining information about a central position of each micro-reaction chamber in the N images;
determining pixels for each micro-reaction chamber in the N images in accordance with a size of each micro-reaction chamber and the information about the central position of each micro-reaction chamber;
determining a fluorescence intensity of each micro-reaction chamber in the N images in accordance with a grayscale value of each pixel for each micro-reaction chamber; and
determining the quantity of positive micro-reaction chambers in accordance with the fluorescence intensity of each micro-reaction chamber in the N images.

5. The biochip detection method according to claim 4, wherein the obtaining the information about the central position of each micro-reaction chamber in the N images comprises:
binarizing an N^{th} image to obtain a binary image;
performing a morphological dilation operation on the binary image in accordance with a first dilation operator in a row direction to obtain a first image, a connected domain in the row direction in the first image representing a row of micro-reaction chambers;
performing a morphological dilation operation on the binary image in accordance with a second dilation operator in a column direction to obtain a second image, a connected domain in the column direction in the second image representing a column of micro-reaction chambers;
detecting connected domains in the row direction in the first image so as to determine the quantity of rows of an array of micro-reaction chambers and information about a central position of each micro-reaction chamber in each row in the column direction;
detecting connected domains in the column direction in the second image so as to determine the quantity of columns of an array of micro-reaction chambers and information about a central position of each micro-reaction chamber in each column in the row direction; and
obtaining the information about the central position of each micro-reaction chamber in accordance with the information about the central position of each micro-reaction chamber in each row in the column direction and the information about the central position of each micro-reaction chamber in each column in the row direction.

6. The biochip detection method according to claim 4, wherein the fluorescence intensity of the micro-reaction chamber is an average of the grayscale values of all pixels for the micro-reaction chambers.

7. The biochip detection method according to claim 4, wherein the determining the quantity of positive micro-reaction chambers in accordance with the fluorescence intensity of each micro-reaction chamber in the N images comprises determining a fluorescence intensity curve of each micro-reaction chamber in accordance with the fluorescence intensity of each micro-reaction chamber in the N images, and determining a micro-reaction chamber whose fluorescence intensity is positively correlated to the intensity of excitation light ray as the positive reaction micro-reaction chamber.

8. The biochip detection method according to claim 5, wherein the binarizing the N^{th} image to obtain the binary image comprises binarizing the N^{th} image using an Otsu algorithm to obtain the binary image.

9. The biochip detection method according to claim 1, wherein the calculating the quantity of copies of the to-be-tested sample in accordance with the quantity of positive micro-reaction chambers comprises calculating the quantity of copies of the to-be-tested sample through c=[ln(1−f/n)]/m, where n is the total quantity of micro-reaction chambers, f is the quantity of positive micro-reaction chambers, m is a dilution factor of the to-be-tested sample, and c is the quantity of copies of the to-be-tested sample.

10. A biochip detection device, comprising:
an introduction module configured to introduce a to-be-tested sample into a biochip, the biochip comprising a plurality of micro-reaction chambers;
an amplification module configured to perform PCR amplification on the to-be-tested sample in the biochip;
an image collection module configured to irradiate the biochip with excitation light rays at different intensities, and collect images of the biochip under the excitation light rays at different intensities, the excitation light rays being used to excite a fluorescent probe in the to-be-tested sample to emit light;
a data processing module configured to perform data processing on the collected images to obtain the quantity of positive micro-reaction chambers; and
a calculation module configured to calculate the quantity of copies of the to-be-tested sample in accordance with the quantity of positive micro-reaction chambers.

11. A biochip detection apparatus, comprising a memory and a processor, wherein a computer program is stored in the memory and executed by a processor so as to implement steps of the biochip detection method according to claim 1.

12. A computer-readable medium storing therein a computer program, wherein the computer program is executed by a processor so as to implement steps of the biochip detection method according to claim 1.
